(19) United States
(12) Patent Application Publication
(10) Pub. No.: US 2004/0219567 A1
(43) Pub. Date: Nov. 4, 2004

(54) Methods for global pattern discovery of genetic association in mapping genetic traits

(76) Inventors: Andrea Califano, New York, NY (US); Aristidis Floratos, Astoria, NY (US); Zhong Li, Rutherford, NJ (US); David G. Wang, Kildeere, IL (US)

Correspondence Address: ROPES & GRAY LLP, ONE INTERNATIONAL PLACE, BOSTON, MA, 02110-2624, US

(21) Appl. No.: 10/703,063

(22) Filed: Nov. 5, 2003

Related U.S. Application Data
(60) Provisional application No. 60/423,849, filed on Nov. 5, 2002.

(51) Int. Cl. C12Q 1/68; G06F 19/00; G01N 33/48; G01N 33/50

(52) U.S. Cl. 435/6; 702/20

(57) ABSTRACT

A pattern discovery-based method for identifying genetic associations in mapping complex traits. In one embodiment, this invention describes the applicable study designs, the pattern discovery algorithm on phenotypic/genotypic data, and methods to evaluate statistical significance of identified patterns. Patterns identified through the proposed methods act as signatures or profiles that can be used to locate genes or genomic regions responsible for the traits of interests in the genome. This invention has been successfully applied to two independent datasets collected from actual genetic studies and has produced significant results.

### REFERENCE TO RELATED APPLICATIONS

This application claims priority to U.S. Provisional Application U.S. Ser. No. 60/423,849, having the same title and naming the same inventors, the contents of which are incorporated by reference.

### BACKGROUND

The systems and methods described herein invention relate to, among other things, methods using genetic information to detect loci for complex traits, such as common human diseases.

Genetic dissection of complex traits has become a major focus for the genetic research community. Lander et al., Initial sequencing and analysis of the human genome, Nature, 2001 Feb. 15; 409(6822):860-921. The completion of the human genome project and the identification of millions of polymorphic markers (Mullikin et al., An SNP map of human chromosome 22, Nature, 2000 Sep. 28; 407(6803):516-20), for the first time in the history of human genetics, make the concept of whole-genome association study (Risch et al., The future of genetic studies of complex human diseases, Science, 1996 Sep. 13; 273(5281):1516-7) to identify disease-causing genes in complex diseases a practical reality. However, as of today, the power of genome-wide association study has yet to be adequately demonstrated, despite of a few, small-scale association studies published recently that have indeed been successful in narrowing down regions where disease is mapped (Rioux et al., Genetic variation in the 5q31 cytokine gene cluster confers susceptibility to Crohn's disease, Nat. Genet., 2001 October., 29(2):223-8; Hugot et al., Association of NOD2 leucine-rich repeat variants with susceptibility to Crohn's disease, Nature, 2001 May 31; 411(6837):599-603; Tavtigian et al., A candidate prostate cancer susceptibility gene at chromosome 17p, Nat. Genet., 2001 Feb., 27(2):172-80).

One critical problem associated with current association studies is that in most cases, the analysis is performed on individual markers or on a small set of markers in a small, contiguous region of the genome. Although single locus analysis is straightforward and the statistics to evaluate significance have been adequately formulated, it is generally believed that it lacks sufficient power to dissect the genetic complexity of common diseases which may have a number of disease-causing loci with small individual effect but whose interaction plays a significant role. Rather, multi-locus analysis seems to provide increased power even with a moderately sized study population (Longmate, Complexity and power in case-control association studies. Am. J. Hum. Genet., 2001 May; 68(5):1229-37) and appears to be the choice for the analysis of complex diseases. Complex diseases may be understood to includes diseases in which more than one gene contributes to, or is involved in the causation, of the phenotype. Complex diseases may be characterized by gene-gene and gene-environment interactions. Examples of complex diseases are Type 2 diabetes, hypertension, and schizophrenia.

There are many outstanding issues with multi-locus analysis for a case-control association study. The first and foremost issue arises from the combinatorial nature of the problem. As the number of possible marker combinations grows exponentially with an increasing number of markers, it quickly becomes impossible to enumerate all combinations within reasonable computational constraints. The second issue associated with multi-locus analysis is the definition of a proper statistical framework. Evaluating the significance of multi-locus contributions to a disease phenotype requires clear definition of the appropriate test and derivation of its statistical properties. Additionally, with the huge number of possible combinations among markers, correction for multiple testing becomes critical to reduce the false positive rate.

Recently, researchers have started to explore interactions among identified disease susceptibility loci through various combinatorial disease modeling (Gabriel et al., Segregation at three loci explains familial and population risk in Hirschsprung disease, Nat. Genet., 2002 May; 31(1):89-93). Statistics have also been proposed (Xiong et al., Generalized T2 test for genome association studies. Am. J. Hum. Genet., 2002 May; 70(5):1257-68) to test significance on multi-locus analysis. However, methods are yet to be developed to efficiently perform multi-locus whole-genome analysis and convincingly assign statistical significance to results, which in turn severely restrict one's ability to take full advantage of the available data.

The systems or methods described herein introduces a novel, global multi-locus genetic data analysis method based on an efficient pattern discovery algorithm and on a framework to assess the statistical significance of patterns, to detect disease-causing loci/genes for complex disease traits. One advantage of this invention has been successfully demonstrated with two independent datasets collected from genome-wide association studies.

### SUMMARY OF THE INVENTION

The invention relates to a pattern discovery-based method for identifying genetic associations in mapping complex traits. In a further aspect, this invention describes the applicable study designs, the pattern discovery process on phenotypic/genotypic data, and methods to evaluate statistical significance of identified patterns. Moreover, patterns identified through the proposed method act as signatures or profiles that can be used to locate genes or genomic regions responsible for the traits of interest in the genome.

One aspect of the invention provides a method for identifying genetic associations through maximal pattern discovery comprising converting information into an appropriate data format; building a seed pattern set with the converted data; and identifying a full pattern from the seed pattern set.

Another aspect of the invention provides a method for identifying genetic associations through maximal pattern discovery comprising converting information into an appropriate data format; computing the number of individuals with a particular marker value for each marker value; and merging patterns from the seed pattern set, merging pairs of patterns in the “emerging pattern set,” and repeating the second merging step until the “combined pattern set” is empty or the number of patterns in the “combined patter set” is larger than a predetermined number.

The method may further comprise partitioning the converted data into subparts. The method may further comprise ordering the seed pattern set, for example, by the linear order of the corresponding markers on the chromosomes. The method may further comprise producing maximal patterns. The method may further comprise quantifying the correlation between the patterns identified and the phenotypic trait under investigation. This quantification may be conducted using chi-square statistics, multi-allele transmission disequilibrium test (TDT), haplotype-based haplotype relative risk (HHRR), statistical significance based on uncorrelated pattern formation, and any other suitable quantification test.

Another aspect of the invention provides a method for selecting a process for maximal pattern discovery wherein the data set contains multiple independent patterns. This method comprises setting the minimum support threshold to full data set size, and performing pattern discovery to identify patterns supported by individuals in a set.

The method may further comprise, if no pattern is found, a process for lowering the minimum support threshold until a predefined minimum pattern is found. To this end the method may comprise masking discovered patterns in the data set and repeating any of the abovementioned steps, for example, setting the minimum support threshold, performing pattern discovery, lowering the minimum support threshold, and/or performing pattern discovery.

To this end, the systems and methods provide, among other things, methods for identifying combinations of two or more loci/genes associated with a detectable phenotype. Given a population of N_{J} individuals (i.e., individuals or chromosomes) sampled over N_{K} markers (i.e., SNPs, microsatellites, etc.), this is accomplished by enumerating the possible combination of at least N_{K0} markers over the possible combination of at least N_{J0} individuals to detect combinations that satisfy certain criteria, (i.e., more likely to occur in individuals with the phenotype than in a control set). This can be accomplished efficiently by using the processes described herein, which avoids exploring the full (N_{K}−N_{K0})! (N_{J}−N_{J0})! possibilities of the complete search space, most of which are typically not realized in the data set.

As described below, the invention can be applied to various genetic study designs such as a population-based association study, a family-based association study, or a traditional linkage study. Depending on the study design, the actual data used by the algorithms may be measured or inferred. For example, in a population-based association study where genotype phase information is unknown, the method can be applied both on the measured genotype data as well as on haplotype data, as produced by any haplotype inference method. A haplotype may be understood as a combination of genotypes on the same chromosome that tend to be inherited as a group. This invention includes the use of biallelic or multi-allelic markers such as single nucleotide polymorphism (SNP) and microsatellite markers, which are polymorphic in the general population as well as in the sub population which a genetic study is sampling. This invention may analyze the distribution of multi-locus alleles/genotypes in individuals expressing the detectable trait and in individuals not expressing the detectable trait. The methods described herein include a process of pattern discovery, typically global pattern discovery, and a process of evaluating statistical significance.

### BRIEF DESCRIPTION OF THE DRAWINGS

The following figures depict certain illustrative embodiments of the invention in which like reference numerals refer to like elements. These depicted embodiments are to be understood as illustrative of the invention and not as limiting in any way.

FIGS. 1A, 1B and 1C present an example of patterns in a simulated genotype dataset;

FIGS. 2A-2D illustrate a complex disease trait and underline patterns explaining each sub-trait;

FIG. 3 presents a flow chart diagram of a global pattern discovery; and

FIG. 4: depicts a flow chart diagram of a process for top down heuristic approach for improving pattern discovery performance.

### DETAILED DESCRIPTION OF CERTAIN ILLUSTRATED EMBODIMENTS

To provide an overall understanding of the invention, certain illustrative embodiments will now be described. However, it will be understood by one of ordinary skill in the art that the systems and methods described herein can be adapted and modified for other suitable applications and that such other additions and modifications will not depart from the scope hereof.

The systems and methods described herein include processes that can analyze large sets of genetic data and identify within the sets patterns that appear to correlate in a statistically significant manner to a genetic trait under investigation. In certain embodiments and practices, the methods apply a global pattern discovery method that allows large sets of genetic data to be subdivided and analyzed separately. Patterns found within the subdivided data sets may be merged to find patterns that occur within large data sets. In this way, the methods described herein provide among other things a computationally feasible process for global pattern discovery thereby providing a pattern discovery process that allows for multi-loci, multi-maker and whole genome pattern discovery and disease association.

Fur purpose of clarity only, the methods and systems described herein will now be described with reference to a process that performs global pattern discovery and statistical data analysis using SNPs found in a sample population.

Global Pattern Discovery

In this embodiment, a dataset of genetic data is collected and analyzed to find one or more patterns.

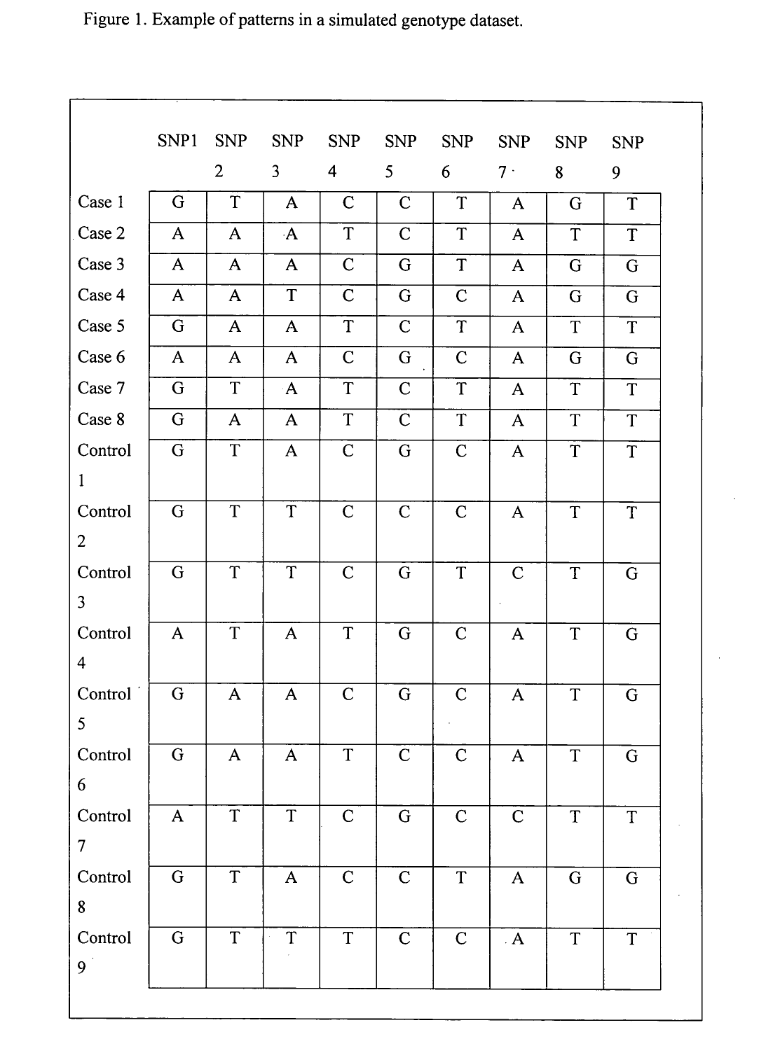

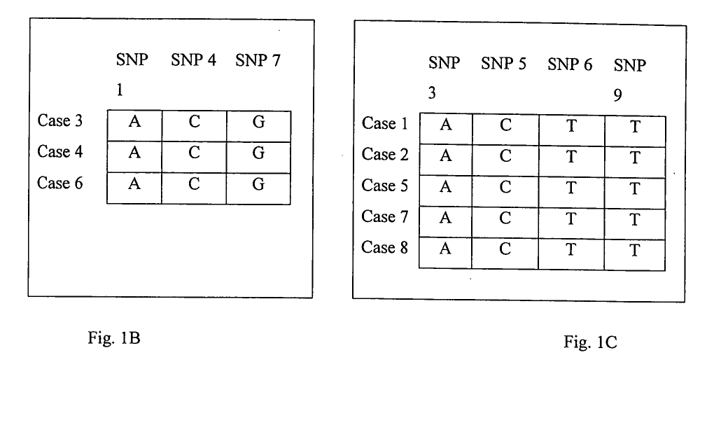

A pattern will be understood to include for this example, although the term is not so generally limited, a set of k markers, M, and j samples, S, such that the set of values of each individual marker across all the j samples fit a predefined similarity criteria (e.g., they all have the same value). Patterns model common recurring features in a dataset. For example, the dataset shown in FIG. 1 contains genotypes from 8 cases and 9 controls based on 9 distinct SNP markers. As described above, a pattern can be treated as a sub-matrix of the full experiment matrix, defined by its support set S (a subset of the individuals) and its marker set M (a subset of the full set of markers over which individuals are genotyped) such that the value of each marker in M fits a predefined similarity criteria across all the individuals in S. In FIG. 1B, for instance, a case-specific pattern (P1) is shown with support IDs S={3, 4, 6} and markers set M={M1, M4, M7}. Another case-pattern (P2) is shown with support IDs S={1, 2, 5, 7, 8} and markers set M={M3, M5, M6, M9}.

In the context of genetic data analysis, a pattern would indicate a collection of one or more features (such as markers) whose values are conserved across a sample (which may be for example set individuals or chromosomes). A pattern can be further classified as an allelic pattern (marker values are alleles) or genotypic pattern (marker values are genotypes). Allelic patterns comprise multi-locus alleles (MLA), while genotypic patterns comprise multi-locus genotypes (MLG). Other types of patterns can be defined and used and the actual pattern employed will depend upon the application at hand with those of skill in the art being free to select the pattern type of interest.

In one practice, the data set (such as the individuals and their genotypes) can be divided into two groups: the “cases” and the “controls”. The first group comprises individuals that have a feature of interest (e.g., they have a disease, or they were submitted to a particular treatment regime) while the second group contains the remaining individuals. When patterns are searched in the context of such a study, the marker set M of a pattern has both a support set, S, relative to the population (A) in which it was discovered (cases or controls), and an incidence set, I, relative to the other population (B). These are the individuals in B for which all the markers in the M set match the corresponding values of the individuals in the S set, based on the similarity criteria. In the previous example of FIG. 1, for instance, the I set for both pattern P1 and P2 is empty. That is, neither pattern has any support in the controls. On the other hand, pattern P3 with S ={1,7} and M={M1, M2} has an incidence set I={1, 2, 3, 8, 9} in the control population.

A pattern may then be described in this practice by three components: (1) The marker set, denoted by M={m₁, m₂, . . . mₖ}, a collection of markers and their values; (2) The support set, denoted by S={s₁, s₂, . . . sⱼ}, a collection of IDs matching the pattern in the first population where it was discovered; (3) The incidence set, denoted by I={i₁, i₂, . . . iⱼ}, a collection of IDs matching the pattern in the second population. The number of markers in a pattern is denoted as NM, while the size of its support and incidence are denoted by N_{S} and N_{I} respectively.

In one practice, the methods described herein seek a set of maximal patterns that exist within the dataset. A maximal pattern in one example will be understood as a pattern such that (1) no marker can be added to the marker set without reducing the size of its support set, j, and (2) no more IDs can be added to the support set without reducing the size of its marker set, k. For instance, in FIG. 1, pattern M={M1, M4} S={P3, P4, P6} is not maximal, because marker M7 can be added without reducing its support S. Reporting non-maximal patterns may result in an overly large set of patterns being generated, and may result in the combinatorial set of sub-patterns being generated for each maximal pattern reported. Furthermore, non-maximal patterns may be less statistically significant than their counterpart maximal patterns. Thus for certain applications processes that report only maximal patterns are desirable because they make the computation more efficient without loss of statistical power. However, the methods described herein contemplate embodiments wherein both maximal and non-maximal patterns can be identified on request, and the choice will depend on the application at hand including such considerations as the overall size of the data set, the computational resources available, the need for certain pattern types, and other such considerations.

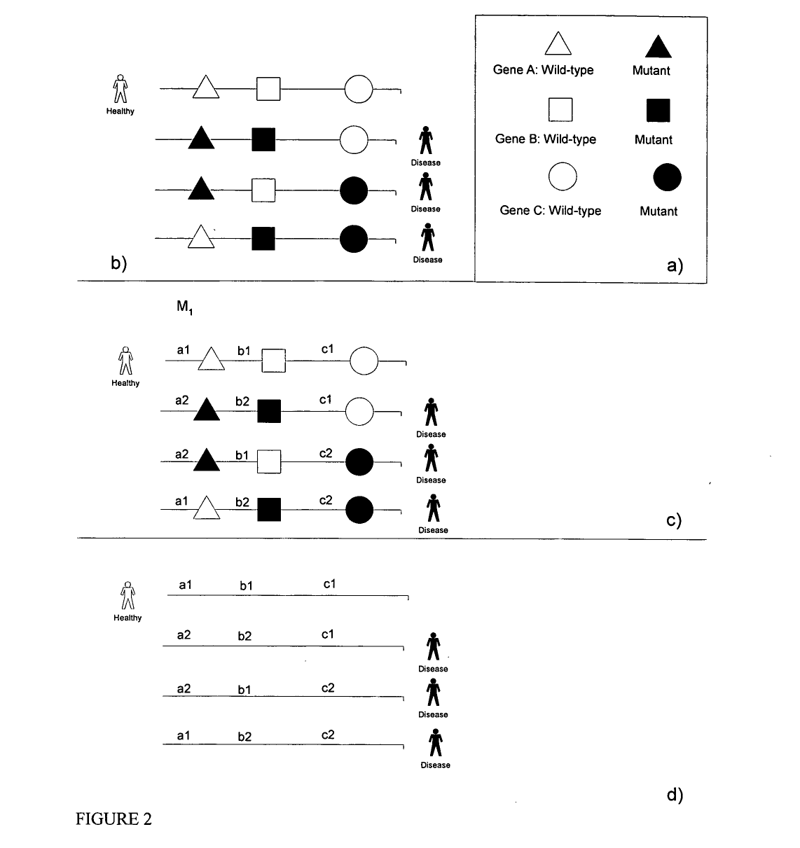

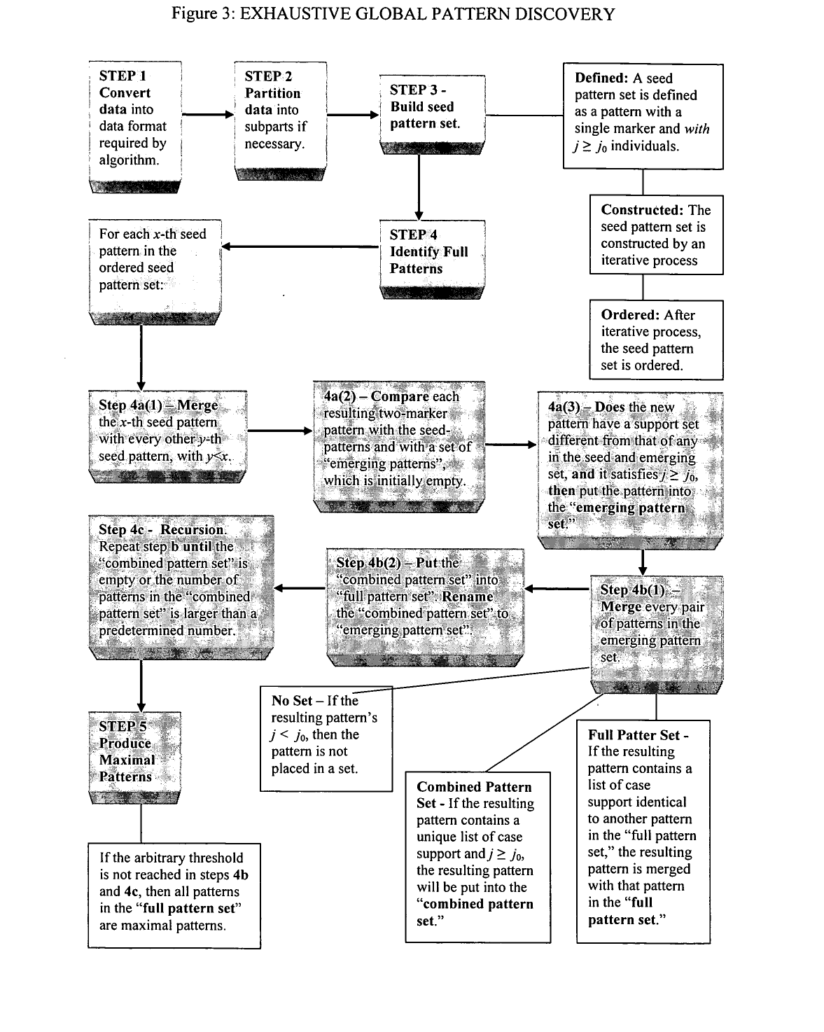

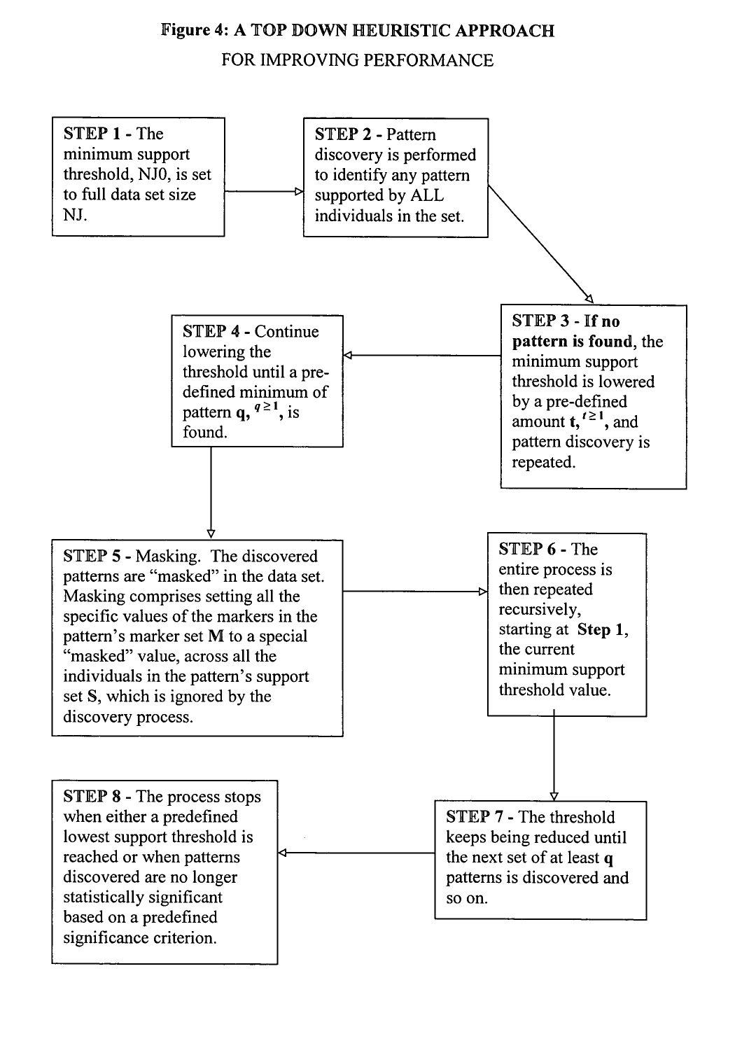

The biological significance of a pattern in genotypic, phenotypic or other data set can be illustrated by the following example. For simplicity of illustration, haploid chromosomes are discussed but the process extends to multiploid organisms. The example addresses a complex disease caused by mutations on several distinct susceptibility genes. For this example, there are three such genes, genes A, B, and C, each one with a benign, wild-type version and a mutant, disease causing version (FIG. 2a). In the case of complex traits disease, phenotypes are usually caused by the interaction of several of the disease genes. For this example, any combination of two mutant variants among the three susceptibility genes, A, B, and C, possibly across multiple chromosomes, can cause the disease as illustrated in FIG. 2b. Furthermore, through appropriate design of the genetic study, the process manages to genotype proximal polymorphic markers that are in linkage disequilibrium with all of the susceptibility genes. Let these markers be M1, M2, and M3 proximal to genes A, B, and C, respectively as illustrated in FIG. 2c. Also assume the following allele assignments:

| Marker | Allele assignments |
|---|---|
| M1 | a1 (wild-type), a2 (mutant bearing) |
| M2 | b1 (wild-type), b2 (mutant bearing) |
| M3 | c1 (wild-type), c2 (mutant bearing) |

In an actual clinical setting the identity/location of the disease susceptibility genes is generally unknown. The data available will be that shown in FIG. 2d, i.e., the values of the markers for each genotyped individual. The ability to localize the genes A, B, and C using the marker data is based on the differential transmission of marker alleles along with the mutant genes.

In such an example, it is probable that none of the three genes has enough individual contribution to the disease phenotype to be identified by single-locus analysis. However, when considering together combinations of markers, the process can identify patterns comprising multiple alleles with significant differential distribution among individuals with disease versus those without the disease. This is realized, given the understanding that the disease manifests itself via the combination of more than one mutant gene. Therefore, patterns provide an efficient model for considering multiple loci together and offer correlations to biological properties. Thus, the systems and methods described herein discover patterns across multiple alleles. However, in other practices and embodiments, the systems and methods described herein may be employed to identify patterns of interest within data sets of regional or local biological data and may be employed for genome-wide association analysis and regional, or local, association analysis.

For any case however, an efficient, deterministic algorithm is described to identify the patterns in a dataset that satisfy the following conditions:

a) The number of markers, k, is equal to or larger than a predefined value k₀. The latter can be set to any value, starting at one.

b) The number of samples,j, is equal to or larger than a predefined value j₀. The latter can be set to any value, starting at two.

c) The patterns are maximal. That is, no additional individual can be added to the pattern without reducing the number of markers and, vice-versa, no additional marker can be added to the pattern without reducing the number of individuals.

d) The pattern satisfies a pre-defined statistical criteria. Several of these will be described in the following sections.

Exhaustive Global Pattern Discovery

As discussed before, two adjustable parameters may be introduced in the pattern-finding algorithm. The first one, k₀, denotes the minimal number of markers in any reported pattern. The second one, j₀, denotes the minimal size of the support set of a reported pattern. The proper use of these parameters provides an efficient controlling mechanism to identify important patterns, associated with a detectable phenotype of trait, without having to report all possible patterns in the data set.

The algorithm may employ a pattern merge operator “+”. The merger operation allows for merging patterns from different dataset—or subsets. For clarity an example is given herein for how two patterns may be merged to create an third distinct pattern. Pattern A (M={m1, m2, m3, m4}, S={s1, s2, s3, s4, s5, s6}) and pattern B (M={m2, m3, m5 }, S={s2, s3, s5, s7, s8}) can be merged to p unique pattern different from either pattern A or B: pattern C=A+B=(M={m1, m2, m3, m4, m5}, S={s2, s3, s5}). The support and marker sets of C are respectively the intersection of the support and marker sets of A and B.

The merger operation may be employed as part of a pattern discovery process that identifies all maximal patterns across a data set of genetic information. One example of such a process is described below.

1. In a first operation, the process converts the original phase-known or phase-unknown genotype data into a data format required by the algorithm. This is akin to the representation in FIG. 1, where each cell, identified by a given individual and marker, is given a value. During this step a decision is made whether to use genotypes or haplotypes. Depending on the original data, haplotypes can be either real or inferred.

2. Partition data into subsets if necessary. Markers can be partitioned according to chromosomes, if necessary, so that patterns can be identified involving a single chromosome instead of all chromosomes. Other criteria, such as selecting markers from candidate gene regions that are likely to interact, are also possible

3. Build “seed pattern set”. A seed pattern is understood as a pattern with a single marker and with j≧j₀ individuals. The “seed pattern set” is constructed by an iterative process. In that process, for each marker value, the number j of individuals with that value is computed. If j≧j₀, then a “seed pattern” is created for that marker and added to the seed pattern set. For example, for a marker M1, marker value A may be observed in cases s1, s2, s3, s4, and s5, and in controls i2, i3, and i4. If j₀=4, for instance, a seed pattern would be formed with the following representation: M={M1}, S={s1, s2, s3, s4, s5}, I={i2, i3, i4}. After all markers are evaluated by this process, seed patterns in the “seed pattern set” are ordered. Many different ways can be used to order seed patterns to facilitate the process described in later steps. One way to order them is by the linear order of their corresponding markers on the chromosomes. All patterns in the “seed pattern set” are put into a “full pattern set”, which is initially empty and is designed to contain all full patterns identified from the dataset.

4. Identify full patterns. In one practice, all combinations of seed patterns in the “seed pattern set” should be represented in the data set. In practice the number of combinations actually present in the data is much smaller and computable, given some practical values of j₀ and k₀. This may be accomplished as follows: for each x-th seed pattern in the ordered “seed pattern set”:

a. Merge that pattern with every other y-th seed pattern, with y<x. That is, only seed patterns with lower ranking than the x-th pattern are considered. Each resulting two-marker pattern may be compared both with the seed-patterns and with a set of “emerging patterns”, which is initially empty. If the new pattern has a support set that is different from that of any in the seed and emerging set, and it satisfies j≧j₀, the pattern is put into the “emerging pattern set”. After seed patterns are processed, the “emerging pattern set” will contain only two-marker patterns with unique support and marker sets. Then each pattern in the “emerging pattern set” is put into the “full pattern set”. Because each pattern in the “emerging pattern set” is different from patterns in the “seed pattern set”, the “full pattern set” maintains that all patterns in the set are unique.

b. Merge optionally every pair of patterns in the “emerging pattern set”. If the resulting pattern of the merging of a pair of pattern in the “emerging pattern set” contains a unique list of case support and j≧j₀, the resulting pattern will be put into the “combined pattern set”, which is empty initially. If the resulting pattern contains a list of case support identical to another pattern in the “full pattern set”, the resulting pattern is merged with that pattern in the “full pattern set”. As the result, the pattern in the “full pattern set” will have more markers in its marker set but maintain the same list of case support. After all pair of patterns in the “emerging pattern set” are evaluated, the resulting patterns are either in the “combined pattern set”, or in the “full pattern set”, or not in any set because j<j₀. The “combined pattern set” is then added into the “full pattern set” and the “combined pattern set” is renamed as the “emerging pattern set” and the new “combined pattern set” is set to be empty.

c. Recursion: Repeat step b until the “combined pattern set” is empty or the number of patterns in the “combined pattern set” is larger than a predetermined number, such as 100,000. The reason for such a threshold is that for a large number of markers with rather homogenous genotypes, single genotype difference in one case support on one marker will produce a huge number of patterns in “the combined pattern set” with very small variations. To set the threshold on the number of patterns in the “combined pattern set”, the algorithm curbs such exponential growth of patterns. The result is that the process no longer identifies all patterns satisfying the initial criteria for j₀ and k₀. Some patterns are missed but the percentage is small in simulations. In return, better efficiency is achieved.

5. Produce maximal patterns. If the arbitrary threshold is not reached in steps b and c, all patterns in the “full pattern set” are maximal patterns. However, because the threshold disrupts the exponential combination of all possible seed patterns, maximality can not be assumed. To ensure that at least all patterns in the “full pattern set” are maximal patterns, the following steps are taken. First, the seed patterns on which the threshold is reached is collected in a “troubled set”. Upon the finish of step c, seed patterns in the “troubled set” are combined in all possible combination to produce a “troubled combined set”. Then the “troubled combined set” is merged with the “full pattern set” in a pair-wise fashion to produce a final set of maximal patterns.

A Top-down Heuristic Approach for Improving Performance

If the data set contains multiple independent patterns, each one with a support set of different size, discovering them all at once can be inefficient. For instance a pattern P1 has the largest support set (e.g., 200) and a pattern P10 has the smallest (e.g., 20). Then, setting the minimum support threshold, NJ0, to 20 would yield trillions of patterns because each possible subset of the support set of P1 could match additional markers from P2 to P10, forming many distinct patterns. Additionally, noise in the data may yield additional pattern breakdown. A top-down method then can be used to recover P1-P10 efficiently. This works as follows. First the minimum support threshold, NJ0, is set to the full data set size NJ. Pattern discovery is performed to identify any pattern supported by ALL individuals in the set. If no pattern is found, the minimum support threshold is lowered by a pre-defined amount t, ^{t≧1}, and pattern discovery is repeated. The threshold keeps being lowered until a pre-defined minimum number of pattern q, ^{q≧1}, is found. Typically q=1 but larger numbers are possible and useful. Then, the discovered pattern(s), or a subset of them identified using statistical significance criteria, are “masked” in the data set. Masking comprises setting all the specific values of the markers in the pattern's marker set M to a special “masked” value, across all the individuals in the pattern's support set S, which is ignored by the discovery process. The process is then repeated recursively, starting at the current minimum support threshold value. The threshold keeps being reduced until the next set of at least q patterns is discovered and so on. The process stops when either a predefined lowest support threshold is reached or when patterns discovered are no longer statistically significant based on a predefined significance criterion. If the patterns are truly independent, this approach allows the efficient identification of each one of them.

Evaluation of Statistical Significance

There are several standard statistical tests are available for quantifying the correlation between the patterns identified in the process described above and the phenotypic trait under investigation. The following is just a sampling of the methods that have been employed:

1. Chi-square statistics. Chi-square statistics can be used to evaluate the distribution bias for a given pattern in case population vs. in control population. In order to accept or reject the null hypothesis of no distribution difference between cases and controls, a contingency table is constructed for each pattern identified in processes described above: The table has two columns, one for case and one for control. The number of rows in the table varies according to the complexity of a pattern. Because a pattern can involve more than one marker, all observed permutations of the pattern where marker alleles/genotypes take different values are treated as their own rows. For example, if a pattern has two markers and 5 different permutations on their genotype values of those two markers are observed in the dataset, the corresponding contingency table will have 5 rows instead of 9 (3²)rows. Each cell of the contingency table holds observed count for a pattern permutation in either case population or in control population. Alternatively, the process can choose to “lump” together the counts of all permutations of the pattern except one (the one that we wish to evaluate). In that case, a contingency table has two rows: one for the specific pattern and one for “all else”, i.e. all the remaining marker value permutations. Regardless of the particular form of the contingency table used, upon calculation of the chi-square statistic, P value is assigned according to default chi-square distribution or to simulated reference distribution through Monte Carlo process, which is described below.

2. Multi-allele Transmission Disequilibrium Test (TDT). TDT is the most widely used method for family-based genetic study (Spielman et al., Transmission test for linkage disequilibrium: the insulin gene region and insulin-dependent diabetes mellitus (IDDM), Am. J. Hum. Genet., 1993 March; 52 (3):506-16), where parents and children in a family are typed. Testing for linkage in the presence of linkage disequilibrium (association), TDT can be very powerful to identify susceptibility locus, especially when the effect is small, as is often the case with complex genetic trait. Although the original TDT test was developed to analyze biallelic markers, new statistics have been developed to accommodate the availability of multiallelic markers or haplotypes (Spielman et al., The TDT and other family-based tests for linkage disequilibrium and asssociation, Am. J. Hum. Gent., 1996 November; 59 (5):983-9; Curtis and Sham, Model-free linkage analysis using likelihoods, Am. J. Hum. Genet., 1995 September; 57(3):703-16; Bickeboller et al., Statistical properties of the allelic and genotypic transmission/disequilibrium test for multiallelic markers, Genet. Epidemiol., 1995; 12(6):865-70). Based on survey performed by Kaplan (Kaplan et al., Power studies for the transmission/disequilibrium tests with multiple alleles, Am. J. Hum. Genet., 1997 March; 60(3):691-702) on those methods, we have chosen the marginal statistics with only heterozygous parents (Tₘₕₑₜ) by Spielman and Ewens (Spielman et al., The TDT and other family-based tests for linkage disequilibrium and association, Am. J. Hum. Genet., 1996 November; 59(5):983-9), because it has equivalent power to the other multi-allelic tests and gives a valid chi-square test of linkage. Multi-allele TDT can be readily applied to patterns because of the multi-allele or multi-genotype nature of a pattern. In a TDT test on a pattern, each observed permutation of a pattern is treated as column and row headings in a TDT contingency table. Corresponding chi-square value is calculated based on described (Spielman et al., The TDT and other family-based tests for linkage disequilibrum and association, Am. J. Hum. Genet., 1996 November; 59 (5):983-9) and P value is assigned according to default or reference distribution simulated by Monte Carlo. This statistics can only be applied to patterns identified in a family-based association study design.

3. Haplotype-based Haplotype Relative Risk (HHRR). HHRR test is another method for family-based studies (Terwilliger et al., A haplotype-based ‘haplotype relative risk’ approach to detecting allelic associations, Hum. Hered., 1992; 42(6):337-46, 1992). It is a variation of the Haplotype Relative Risk (HRR) method, which is genotype-based. In Rubinstein's Genotype-based haplotype relative risk (GHRR) method, the affected children's genotypes at a marker locus are used as cases and artificial genotypes made up of the alleles not transmitted to the children from their parents are used as controls. For each haplotype of interest, a 2×2 contingency table is constructed and used to record the number of cases and controls with or without that haplotype. In contrast, HHRR utilizes haplotypes rather than genotypes. In particular, transmitted chromosomes are treated as cases and untransmitted chromosomes are used as controls, A 2×2 table is constructed the same as for GHRR. HHRR can be extended to be applied to patterns because of the similarity between a pattern and a multi-marker haplotype. In a HHRR test for a pattern, the observed counts for the pattern in cases and in controls and the observed counts for all other permutations on markers in that pattern in cases and controls are recorded in the 2×2 contingency table. Upon the calculation of chi-square values, P values are assigned according to default distribution or reference distribution simulated by Monte Carlo.

4. Statistical significant based on uncorrelated pattern formation (Califano et al., Analysis of gene expression microarrays for phenotype classification, Proc. Int. Conf. Intell. Syst. Mol. Biol., 2000; 8:75-85).

Monte Carlo simulation is used to derive proper significance level for calculated chi-square values. In each round of Monte Carlo iteration, the case/control category assignment for each actual case/control id is randomized. As the result, the simulated case dataset will contain both actual case genotypes/haplotypes and control genotypes/haplotypes. The Pattern discovery process is then applied on simulated case dataset. After deriving chi-square value for each identified patterns in the case dataset with the procedure described above, the chi-square value and the corresponding degree of freedom are recorded. After all iterations, chi-square distributions are plotted with frequencies of chi-square values for each degree of freedom.

Patterns selected as being statistically relevant may then optionally be further explored and analyzed, such as by wet lab testing including animal models and yeast tests. Thus, it will be understood that the processes described above may be used to identify target genes and collections of genes/loci for further analysis and for disease association.

In another aspect, it will be understood that the invention provides systems that may be employed to identify patterns with a data set. The systems may be machines as well as software tools and can include devices for processing sequence data as well as data visualization tools which can highlight patterns in data that is visually displayed. The system may comprise a conventional data processing platform such as an IBM PC-compatible computer running the Windows operating systems, or a SUN workstation running a Unix operating system. Alternatively, the system can comprise a dedicated processing system that includes an embedded programmable data processing system. For example, the system can comprise a single board computer system that has been integrated into a system for sequencing genomic data, identifying SNPs or markers, collecting expression data, or for performing other laboratory processes.

The processes and systems described above and illustrate in the Figures can be realized as a software component operating on a conventional data processing system such as a Unix workstation. In that embodiment, the process can be implemented as a C language computer program, or a computer program written in any high level language including C++, Fortran, Java or Basic. Additionally, in an embodiment where microcontrollers or DSPs are employed, the process can be realized as a computer program written in microcode or written in a high level language and compiled down to microcode that can be executed on the platform employed. The development of such systems is known to those of skill in the art, and such techniques are set forth in Digital Signal Processing Applications with the TMS320 Family, Volumes, I, II, and III, Texas Instruments (1990). Additionally, general techniques for high level programming are known, and set forth in, for example, Stephen G. Kochan, Programming in C, Hayden Publishing (1993). It is noted that DSPs are particularly suited for implementing signal processing functions, including preprocessing functions such as image enhancement through adjustments in contrast, edge definition and brightness. Developing code for the DSP and microcontroller systems follows from principles well known in the art.

Those skilled in the art will know or be able to ascertain using no more than routine experimentation, many equivalents to the embodiments and practices described herein. For example, the systems and methods described herein may be employed in other applications including financial applications, engineering applications and other applications that would benefit from having patterns found within a large dataset. Accordingly, it will be understood that the invention is not to be limited to the embodiments disclosed herein, but is to be understood from the following claims, which are to be interpreted as broadly as allowed under the law.

1. A method for genetic data analysis, comprising the steps of
collecting genetic data from a plurality of subjects, setting two adjustable parameters for controlling a pattern discovery process;
applying a pattern discovery process as a function of the adjustable parameters to find shared features in the genetic data and to identify a set of seed patterns, and
applying a statistical test which assigns a significance value to respective ones of the seed patterns representative of whether the respective seed pattern qualifies for further analysis.

2. A method according to claim 1, further comprising
determining the statistical significance of a respective seed pattern.

3. A method according to claim 2, wherein determining the statistical significance includes comparing against a null hypothesis.

4. A method according to claim 2, wherein the null hypothesis comprises
determining a measure representative of the strength of representation of a pattern in a control population.

5. A method according to claim 1, wherein
collecting genetic data includes collecting sequence data.

6. A method according to claim 1, wherein
collecting genetic data includes collecting data from the group consisting of genotypic data, haplotype data, allelic data, and phenotypic data.

7. A method according to claim 1, wherein
collecting genetic data includes collecting data from a case population consisting of individuals having an indication of interest.

8. A method according to claim 1, wherein
collecting genetic data includes collecting data from a control population consisting of individuals selected from a general population.

9. A method according to claim 1, wherein applying a statistical test includes comparing a distribution bias associated with a seed pattern found from data associated with a case population to a distribution bias associated with a seed pattern found from data associated with a control population.

10. A method according to claim 1, wherein applying a statistical test includes comparing a distribution bias associated with a seed pattern found from data associated with a case population to a null hypothesis developed according to a statistical analysis of a control population.

11. A method according to claim 1, wherein selecting two adjustable parameters includes selecting a parameter representative of a minimum number of markers in a pattern and a minimum number of samples having the pattern.

12. A method according to claim 1, wherein applying a pattern discovery process includes sorting genetic data representative of markers found for members of a population to identify a pattern of one or more markers that is associated with a predetermined minimum number of population members.

13. A method according to claim 1, further comprising
merging patterns found from multiple datasets to generate extended patterns.

14. A method according to claim 1, further comprising
sorting through identified seed patterns to find maximal patterns representative of patterns constrained by a marker criteria and a support population criteria.

15. A method according to claim 1, wherein
discovered patterns are employed in disease association analysis.

16. A method according to claim 1, wherein
discovered patterns are employed in linkage analysis.

17. A method according to claim 1, wherein
discovered patterns are employed in family-based genetic analysis.

18. A method according to claim 1, wherein
discovered patterns are employed in population-based genetic analysis.

19. A method according to claim 1, wherein
discovered patterns are employed in sib-pair study analysis or family-trio study analysis.

20. A method according to claim 1, wherein applying a statistical test applying a non-statistic test includes calculating the odds ratio.

21. A method according to claim 1, wherein
discovered patterns are employed in genome-wide association analysis.

22. A method according to claim 1, wherein
discovered patterns are employed in regional association analysis.

23. A method according to claim 1, wherein
the data set includes 1 or more markers.

24. A method according to claim 1, wherein
applying a statistical test includes a test selected from the group chi-square,
Fisher's exact test, transmission disequilibrium test (TDT), haplotype-based haplotype relative risk (HHRR), and T-Test.

25. A method according to claim 1, employed to detect locus/gene interactions between two or more loci.

26. A method according to claim 1, employed to detect genetic heterogeneity, population substructure or to detect multi-locus association, in which each locus only has small-moderate effect.

27. Apparatus for genetic data analysis, comprising
a database having genetic data from a plurality of subjects,
a process for setting two adjustable parameters for controlling a pattern discovery process, and applying a pattern discovery process as a function of the adjustable parameters to find shared features in the genetic data and to identify a set of seed patterns, and
a statistical test process for assigning a significance value to respective ones of the seed patterns representative of whether the respective seed pattern qualifies for further analysis.

28. An apparatus according to claim 27, further comprising
a process for determining the statistical significance of a respective seed pattern.

29. A method for identifying genetic associations through maximal pattern discovery, comprising
collecting genetic information from a plurality of patients,
converting the genetic information into a predetermined data format;
building a seed pattern set with the converted data; and
identifying a full pattern from the seed pattern set by applying at least two constraints to the full patterns.

30. A method for identifying genetic associations through maximal pattern discovery across multiple data sets, comprising
collecting genetic information from a plurality of patients,
converting the genetic information into an appropriate data format;
computing the number of individuals with a particular marker value for each marker value;
merging patterns from a seed pattern set;
merging pairs of patterns in an emerging pattern set, and repeating the second merging step until a combined pattern set is empty or the number of patterns in the combined pattern set is larger than a predetermined number.

31. A method according to claim 1 further comprising
providing a data set that contains multiple independent patterns setting a minimum support threshold for full data set size; and
performing pattern discovery to identify patterns supported by individuals in a set.
